(19) United States
(12) Patent Application Publication
(10) Pub. No.: US 2004/0072754 A1
(43) Pub. Date: Apr. 15, 2004

(54) Amyloid precursor protein and APP-deprived peptides inhibit tumor growth and metastasis

(76) Inventors: Moshe Marikovsky, Mazkeret Batya (IL); Yoram Groner, Rehovot (IL); Catherine Harris-Cerruti, Rehovot (IL)

Correspondence Address: WINSTON & STRAWN, PATENT DEPARTMENT, 1400 L STREET, N.W., WASHINGTON, DC, 20005-3502, US

(21) Appl. No.: 10/423,047

(22) Filed: Apr. 25, 2003

Related U.S. Application Data
(63) Continuation of application No. PCT/IL01/00986, filed on Oct. 25, 2001.

(30) Foreign Application Priority Data
Oct. 26, 2000 (IL) IL 139308

(51) Int. Cl. A61K 38/17; A61K 38/10; A61K 38/08

(52) U.S. Cl. 514/12; 514/13; 514/14; 514/15; 514/16; 514/17

(57) ABSTRACT

Amyloid precursor protein and non-glycosylated peptides derived therefrom especially from the Aβ domain of amyloid precursor protein are useful in prevention or treatment of cancer, and for immunostimulation in individuals with compromised immune systems. A peptide comprising residues 1-42 of the amyloid precursor protein, as well as smaller fragments and analogs of this peptide, which demonstrate the anti-cancer activity, are disclosed. A peptide comprising residues 1-16 of the amyloid precursor protein is one currently preferred active fragment. Pharmaceutical compositions comprising these peptides, and methods of using them to prevent or inhibit tumor growth and metastases are disclosed. Methods of gene therapy using APP or APP-derived peptides are also disclosed for treatment of cancer and for immunostimulation.

### CROSS-REFERENCE TO RELATED APPLICATIONS

This application is a continuation of the U.S. National Stage designation of International Application PCT/IL01/00986, filed on Oct. 25, 2001, which included a claim of priority to IL Application No. 139308, filed Oct. 26, 2000, which are expressly incorporated herein in their entireties.

### FIELD OF THE INVENTION

This invention relates generally to amyloid precursor protein and peptides derived therefrom, and more particularly to pharmaceutical compositions comprising them. The invention also relates to methods for treating and/or preventing cancer and methods for immunostimulation.

### BACKGROUND OF THE INVENTION

The amyloid precursor proteins (APP) comprise a group of ubiquitously expressed transmembrane glycoproteins whose heterogeneity arises from both alternative splicing and post-translational processing [Selkoe, D. J. (1994)]. Apart from the 751- and 770-residue splice forms expressed in non-neuronal cells throughout the body, neurons express a more abundant 695-residue isoform. All isoforms are the precursors of various metabolites that result from different proteolytic cleavage induced by physiological or pathological conditions.

Recent evidence suggests that aberrant processing of APP may be causally related to the neuronal degeneration that occurs in Alzheimer's disease. Due to its possible involvement in neurodegeneration there has been considerable research into the function and processing of the APP. Despite this widespread interest, the principal function(s) of the molecule in vivo remain unclear.

APP contains a globular domain (containing heparin-, zinc-, and copper-binding domains), an acidic domain, a Kunitz-type of serine protease inhibitor (KPI) domain (present only in the long 751 and 770 isoforms) and a glycosylation domain that may be involved in dimerization [for review see Li, Q. X. et al. (1999)].

Functions for APP that have been described in vitro include enhancement of cell-substrate adhesion, neuritotrophic and other growth promoting effects and neuroprotective properties [Selkoe, D. J. (1994)].

APP is ubiquitously expressed, albeit in multiple alternative forms. In the rat, testis, ovary, liver, spleen, pancreas and salivary gland were immunostained. APP is highly expressed in Sertoli cells, follicle cells, secretory cells, podocytes and macrophages [Beer, J. et al. (1995)], as well as in pituitary and adrenal glands, in cardiac muscle [Arai, H., et al. (1991)] and in thyroid epithelial cells which produce large amount of APP and also the Aβ peptide [Schmitt, T. L., et al. (1995)]. Platelets are the primary source in the circulation, producing greater than 90% of the circulating APP or of Aβ. Low concentrations (10 pM) of carboxyl-terminally truncated APP-KPI⁺are found in plasma when blood is carefully collected with minimal platelet activation. Although the origin is uncertain, studies suggest that the major source may be platelets due to their high concentration of APP (30 nM) compared to other cells in the circulation. Turnover rates of Aβ and APP are 2 hr and 7 hr correspondingly [Li, Q. X., et al (1999)].

Alzheimer disease (AD) is the most common cause of progressive cognitive decline in the aging human population. The main pathological lesions of AD consist of the extracellular deposits of amyloid in the brain in the form of plaques and congophilic angiopathy, as well as intracellular neurofibrillary tangles. The amyloid consists mostly of the self-aggregating Aβ and the smaller p3 peptides, both of which are proteolytically derived from APP. The Aβ region spans 40-43 residues and is located in the juxta-membrane domain of APP. Among the Aβ peptides formed under normal conditions, approximately 90% of secreted AD consists of Aβ₁-40 peptide and about 10% consists of longer Aβ_{1-42/43} peptides. Although the Aβ_{1-42/43} peptides are minor Aβ products, these longer Aβ peptides are more amylogenic than the shorter peptides, and are thought to initiate AP deposition and plaque formation [Li, Q. X., et al (1999)]. Diffuse deposits are almost exclusively composed of the highly amyloidogenic Aβ 1-42. The diffuse deposits are not correlated with the clinical manifestations of AD since they exist in regions that are generally not implicated in clinical symptoms. Likewise, the brains of aged cognitively normal humans often contain Aβ deposits, but these are primarily of the diffuse type, with few neuritic plaques and neurofibrillary tangles present in limbic and association cortices [Selkoe, D. J. (1999)]. Aβ also accumulates in the basement membrane of some cerebral capillaries, arterioles and venules and some meningeal arterioles. The extent of this microvascular β-amyloidosis usually does not correlate closely with the number of Aβ plaques in a brain, and its importance in contributing to the dementia is unclear [Selkoe, D. J. (1999)]. Neurofibrillary tangles are intraneuronal cytoplasmic lesions consisting of non-membrane-bound bundles of paired, helically wound 10-nm filaments (PHF). Neurofibrillary tangles generally occur in large numbers in the Alzheimer brain. There is growing evidence that the formation of tangles represents one of cytological responses by cells to the gradual accumulation of AD and Aβ associated proteins [Selkoe, D. J. (1999)]. Aβ production appears to occur in all cells and tissues of the body, however, only Aβ depositions in the brain are associated with Alzheimer's pathology. This suggests that other factors specific to the CNS may be involved in promoting the Aβ deposition and/or preventing its clearance.

Tranisgenic mice overexpressing the three human isoforms of APP in the brain, did not show the pathological changes associated with AD and Down's Syndrome (DS). However they demonstrated significant changes in spatial navigation tasks and motor behavior [Czech, C. et al (1994)]. Tangles have not been described in existing APP mouse models. It is the expression of ApoE4 or mutations in presenilin that result in increased aggregation of cerebral AD, elevation in Aβ₁₋₄₂ and accelerated AD-like phenotype [Selkoe, D. J. (1999)].

In vitro studies were conducted to define the Aβsequence responsible for neurotoxicity. The Aβ₂₅₋₃₅, Aβ₁₋₃₈ or Aβ₁₋₄₀ induced toxicity in hippocampal cell cultures while Aβ₁₋₁₆ and Aβ₁₋₂₈ were non-toxic to neuronal cells [Iversen et al., 1995]. Aβ₂₅-35 region was shown to enhance aggregation and fibrillar formation as well as neurotoxicity in vitro at 25 uM concentrations [Pike et al., 1995].

While these APP-derived peptides of the Aβ domain have been extensively studied for their possible neurotoxic effects, there is no teaching or suggestion that they may be beneficial or of therapeutic utility.

U.S. Pat. No. 5,550,216 discloses a gelatinase A inhibitor comprising as an active ingredient a peptide analogue consisting of an active minimum unit of gelatinase A inhibition obtained from APP (β-amyloid precursor) or a peptide analogue comprising it.

Gelatinase A is known also as one of the matrix metalloproteinases secreted by cancer cells and the like. Gelatinase A is also suspected to promote local destruction of the tissues which occurs in the process of infiltration and metastasis of cancer or to promote migration of leukocytes during inflammation. Thus, it is expected that inhibition or suppression of the activity of gelatinase A may ameliorate cancer metastasis or inflammatory diseases. According to that disclosure, the active minimum unit of inhibition of gelatinase A activity is regarded as a peptide analogue to which carbohydrates are bound and which consists of 439 V through 687 K of the amino acid sequence of APP770.

Nowhere in the background art is it taught or suggested that APP or fragments of APP other than the gelatinase inhibitory fragment, and especially non-glycosylated fragments of less than 100 amino acids are useful in preventing metastasis or treating cancer.

### SUMMARY OF THE INVENTION

It is now disclosed that amyloid precursor protein itself, and especially small peptide fragments of no more than 100 amino acids derived from the intact protein are unexpectedly useful in the treatment or prevention of cancer and metastasis. Although the invention is exemplified herein with certain currently preferred embodiments comprising specific peptides of the AP domain in APP, it is disclosed herein that the intact APP as well as additional non-glycosylated fragments of the APP protein are suitable for use in accordance with the principles of the invention as anti-cancer therapeutic agents. All of the alternative splice forms of the APP are useful for the purposes of the invention, including the 751- and 770-residue splice forms expressed in non-neuronal cells throughout the body, as well as the 695-residue isoform expressed by neurons.

The currently preferred peptides according to the invention are peptides from the AP domain in APP. Currently most preferred peptides according to the invention include:

Peptide 1-42 (Sequence ID No.1):

Asp-Ala-Glu-Phe-Arg-His-Asp-Ser-Gly-Tyr-Glu-Val-His-His-Glri-Lys-Leu-Val-Phe-Phe-Ala-Glu-Asp-Val-Gly-Ser-Asn-Lys-Gly-Ala-Ile-Ile-Gly-Leu-Met-Val-Gly-Gly-Val-Val-Ile-Ala (672 D through 713 A of the amino acid sequence of APP770)

and Peptide 1-16 (Sequence ID No.2):

Asp-Ala-Glu-Phe-Arg-His-Asp-Ser-Gly-Tyr-Glu-Val-His-His-Gln-Lys (672 D through 687 K of the amino acid sequence of APP770) of the Aβ domain in APP.

Pharmaceutical compositions comprising as an active ingredient a therapeutically effective amount of a non-glycosylated fragment of APP are disclosed. It is to be understood that the non-glycosylated peptides of the present invention encompass non-glycosylated peptides from the glycosylation domain as well as any other active fragment of APP.

The Amyloid Precursor Protein itself, as used according to the principles of this invention, can be any of the alternative splice forms of this molecule and may be used either as a glycosylated or non-glycosylated form.

According to the principles of the present invention use of non-glycosylated peptides derived from APP for the manufacture of medicaments for the treatment and prevention of tumor growth or metastasis are disclosed.

Methods of treating an individual, preferably a human with a therapeutically effective amount of a non-glycosylated peptide derived from APP are disclosed, as are suitable regimens for the prevention of tumor growth or metastasis.

Gene therapy may be performed with vectors or cells bioengineered to express APP, or APP-derived peptides. More advantageously the cells may be autologous cells of the individual who is in need of the treatment. The cells bioengineered to express APP may be peripheral blood lymphocytes (PBL), stem cells, including but not limited to bone marrow derived stem cells or other cell types of the patient. Alternatively and advantageously it may also be possible to use pluripotent human embryonic stem cells as a suitable population for gene therapy. Alternatively and advantageously, gene therapy may be performed either in vivo or ex-vivo using a variety of appropriate vectors, by many appropriate methods as known in the art.

Within the scope of the present invention it is further contemplated that treatment with APP, or APP-derived peptides is also useful in circumstances where the immune system is compromised. Immunodeficiency whether innate or acquired, as well as states in which the immune system is temporarily compromised due to chemotherapy or following transplantation of bone marrow, may be beneficially treated by APP or APP-derived peptides. Gene therapy using either vectors or cells bioengineered for overexpression of APP or APP-derived peptides may also be useful for treatment of states of immunodeficiency.

### BRIEF DESCRIPTION OF THE DRAWING FIGURES

FIG. 1: Lewis lung carcinoma development is completely inhibited in Tg-mice Metastatic growth in the lungs of Tg-SOD/APPH, Tg-APPH and control parental mice (APPN) was evaluated. The weight of lungs derived from normal mice was subtracted from that of the metastasized lungs in both models (n=9).

FIG. 2: Lewis lung carcinoma development is completely inhibited in Tg-APPH mice. Metastatic growth in the lungs of Tg-APPH and control parental mice (APPN) was evaluated by: A. Photography and B. Measuring lung weight. The weight of lungs derived from normal mice was subtracted from that of the metastasized lungs in both models. (n=12).

FIG. 3: Lewis lung carcinoma development is completely inhibited in Tg-APPH mice in a long-term experiment. Metastatic growth in the lungs of control parental (APPN) and Tg-APPH mice was evaluated by measuring lung weight after 27-36 days or 136 days, correspondingly. The weight of lungs derived from normal mice was subtracted from that of the metastasized lungs in both models. (APPN, n=10; Tg-APPH, n=14).

FIG. 4: Heterozygous Tg-APP mice display an intermediate resistance to metastasis. Metastatic growth in the lungs of Tg-APPH, control parental mice (APPN) and heterozygous Tg-APP mice was evaluated by metastases count. (n=10).

FIG. 5: Lewis lung carcinoma tumor development in the footpad is inhibited in transgenic mice homozygous for APP (Tg-APPH mice). The size of primary tumors in Tg-APPH and control parental mice (APPN) was evaluated by measuring the width of the footpad (n=9).

FIG. 6: Aβ₁₋₄₂ peptide inhibits Lewis lung carcinoma metastasis. Metastatic growth in the lungs of C57/BL mice was evaluated by A. Photography and B. Metastases counts (n=10).

FIG. 7: Effect of various Aβ peptides on Lewis lung carcinoma metastasis. C57/BL mice were injected intraperitoneally with the Aβ₁₋₄₂, Aβ₁₋₆₃ Aβ₁₀₋₂₀ and Aβ₉₆₋₁₁₀ peptides. Metastatic growth was evaluated by A. Photography and B. Counting metastases (n=7).

FIG. 8: Scheme of the structure of Amyloid precursor protein (according to Li et al. 1999).

### DETAILED DESCRIPTION OF THE PREFERRED EMBODIMENTS

Unexpectedly, it has now been discovered that overexpression of amyloid precursor protein is effective in reducing the susceptibility of animals to primary tumors or metastases. It is now disclosed that administration of certain non-glycosylated peptides derived from amyloid precursor protein is effective in the prevention and treatment of tumor growth and metastasis. The active peptides are derived from the AP domain of APP, and comprise approximately 5 to 100 amino acid residues in length. More preferably the active peptides comprise about 10-50 amino acid residues, most preferably the active peptides comprise 15-45 amino acid residues.

It will be realized by the artisan that the present invention is operative with the peptides disclosed either individually or in combination therapy. Combination therapy may entail administering mixtures of a plurality of peptides. In the alternative it may involve administering more than one type of pharmaceutical composition of individual peptides to the same subject undergoing treatment.

While peptides derived from APP are known in the art there is no teaching of non-glycosylated peptides in general or of peptides of less than 100 amino acids in particular being useful in the treatment or prevention of cancer. According to the present invention a useful peptide may be derived from any domain of the molecule other than the isolated glycosylation domain bearing at least one carbohydrate moiety, previously shown to possess gelatinase inhibitory activity. According to the present invention the non-glycosylated peptide may also be a non-carbohydrate bearing peptide derived from the glycosylation domain.

The useful peptides may be derived from the APP globular domain, acidic domain, KPI domain, as well as the Aβ region. Non-glycosylated peptides derived from the glycosylation domain may also be used according to the present invention.

Unexpectedly, it was found that Transgenic (Tg) mice over-expressing the amyloid precursor protein (APP) either alone or together with Cu/Zn superoxide dismutase (SOD-1) were shown to be completely resistant to development of Lewis lung carcinoma in the lungs. Moreover, primary tumors in Tg-APP mice were reduced by 90% compared to control parental mice. These transgenic mice may be rendered resistant to tumor growth due to the presence of elevated levels of intact APP. It is also possible that peptides derived from the overexpressed APP provide the anti-cancer activity. Accordingly, certain commercially available peptides were tested for their anti-cancer activity when administered individually to host animals in which tumors were induced.

External administration to mice of two specific peptides comprising part of the Aβ domain in APP (1-42, 1-16) is now disclosed to considerably lower the metastatic load of Lewis lung carcinoma in these mice (66-81%).

While the invention will now be described in connection with certain preferred embodiments in the following figures and examples so that aspects thereof may be more fully understood and appreciated, it is not intended to limit the invention to these particular embodiments. On the contrary, it is intended to cover all alternatives, modifications and equivalents as may be included within the scope of the invention as defined by the appended claims. Thus, the following figures and examples which include preferred embodiments will serve to illustrate the practice of this invention, it being understood that the particulars shown are by way of example and for purposes of illustrative discussion of preferred embodiments of the present invention only, and are presented in the cause of providing what is believed to be the most useful and readily understood description of formulation procedures as well as of the principles and conceptual aspects of the invention.

### EXAMPLES

Materials and Experimental Systems

Aβ₁₋₁₆, Aβ₁₋₄₂ Aβ₉₆₋₁₁₀ and Aβ₉₆₋₁₁₀ peptides were purchased from either Sigma (St Louis, Mo., USA) or Bachem AG (Bubendorf, Switzerland). These peptides are disclosed herein as Sequence ID Nos. 1-4.

Aβ₁₀₋₂₀ (Sequence ID No.3) has the sequence Tyr-Glu-Val-His-His-Gln-Lys-Leu-Val-Phe-Phe and Aβ₉₆₋₁₁₀ (Sequence ID No.4) has the sequence Asn-Trp-Cys-Lys-Arg-Gly-Arg-Lys-Gln-Cys-Lys-Thr-His-Pro-His.

Recombinant basic fibroblast growth factor (b-FGF) was kindly provided by Prof. Gera Neufeld, (Technion, Haifa, Israel).

Cells. The metastatic clones D122 and B16-F10 melanoma, were kindly provided by Prof. L. Eisenbach, (Weizmann Institute, Rehovot, Israel) and were used for cell culture and in vivo experiments as described [Eisenbach, L. et al. (1983); Mandelboim, O. et al (1995); Porgador, A. et al., (1989)].

Tg-Mice. Transgenic mice harboring the human APP gene (Lamb, B. T. et al. 1993) and homozygous for the transgene were bred and used for the experiments. Mice genotypes were determined either by Southern blotting or genomic PCR, distinguishing between wild type and transgene. Human APP transcripts and protein were detected in the mice at levels similar to the endogenous mouse products in the brain and in other tissues. Homozygous Tg-APPH mice carry two copies of the native human APP gene and levels of APP protein were elevated three fold over that found in control non-transgenic mice.

Homozygous Tg-SOD animals overexpress the native human Cu/Zn superoxide dismutase entire gene with its own promoter [Avraham, K. B. et al (1988)] and present a 3-fold increase of the enzyme activity in the brain versus control mice. The presence and activity of human SODI transgene were determined by genomic PCR and by SODI enzymatic assay applied to blood samples.

Hieterozygous APP mice were developed by inbred mating or by back-crossing between Tg-APPH and control parental mice.

Double Tg-mice SOD1/APP were developed by inbred mating between Tg-SOD and Tg-APPH. All experiments were carried out with male Tg-mice and age-matched control mice. All mice were housed in a pathogen free environment under standard conditions and maintained on a 12:12 hour light/dark cycle with food and water ad libitum.

Metastasis of Lewis Lung Carcinoma and B16 Melanoma Tumors to the Lungs.

The Lewis lung carcinoma (3LL), which originated spontaneously in a C57/BL/6J(H-2^{b}) mouse, is a malignant tumor that produces spontanfeous lung metastases [Eisenbach, L. et al. (1983); Mandelboim, O. et al (1995)]. The B16-F1O melanoma is another established tumor cell line derived from C57BL mice useful for assessing metastatic potential as it is a highly metastatic tumor that produces spontaneous lung metastases [Porgador et al., 1989].

Two models of metastasis were used: 1. The footpad model. 2. The intravenous (i.v.) model. The assay of tumor development in the footpad and evaluation of lung metastases was performed as described [Eisenbach, L. et al. (1983); Mandelboim, O. et al (1995)]. D122 or B16 tumor cells were injected i.v. into Tg-APP, Tg-APP/SOD or parental mice and metastasis was evaluated after 30-37 days. In certain long-term experiments the evaluation was performed over a period of 120-140 days post injection of the tumor cells.

Aβ peptides in phosphate buffered saline (PBS) were injected i.p. into C57/BL mice 4-7 times (7-10 μg each time or 10-20 μg each time in other experiments) during two weeks preceding D122 or B16 tumor cells injection and seven times every second day following D122 or B16 tumor cells injection. Control mice were i.p. injected with PBS. Mice were sacrificed 20-21 days following injection of D122 or B16 tumor cells by injecting 20 mg/mouse Xylazine (i.p.) and lungs were weighed. Lungs were fixed, stained in Bouin's solution and metastases counted. For histology, lungs were fixed in buffered formalin and histological sections were prepared and stained with Hematoxylin-Eosin. Each experimental group included 7-12 animals, and experiments were repeated twice.

Statistical analyses. Following analysis of variance 2 tailed-p Student t-test was applied.

### Example 1

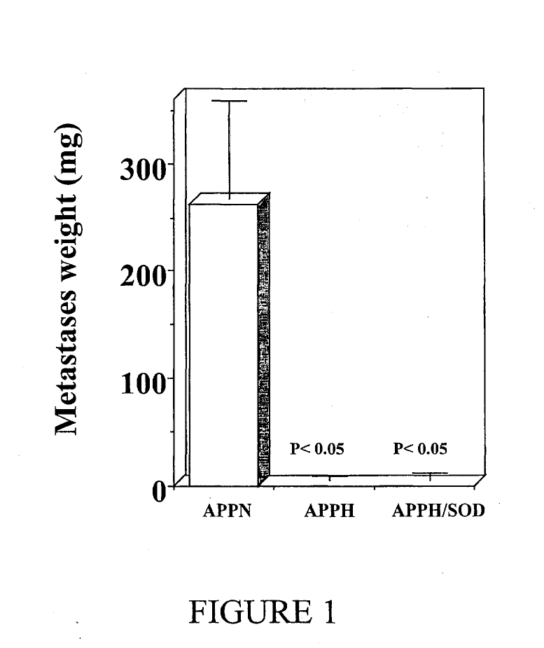

Lewis lung carcinoma development is completely inhibited in Tg-mice. D122 tumor cells were injected i.v. to the tail of transgenic mice harboring the human Cu/Zn SOD and APP gene (Tg-SOD/APPH), to transgenic mice harboring the human APP gene (Tg-APPH) and to control parental mice (APPN). After 30 days the lungs were examined and weighed. No metastases whatsoever were found in the lungs of Tg-SOD/APPH or in those from Tg-APPH, while the lungs of control APPN mice were heavily loaded with metastases (260 mg) (FIG. 1).

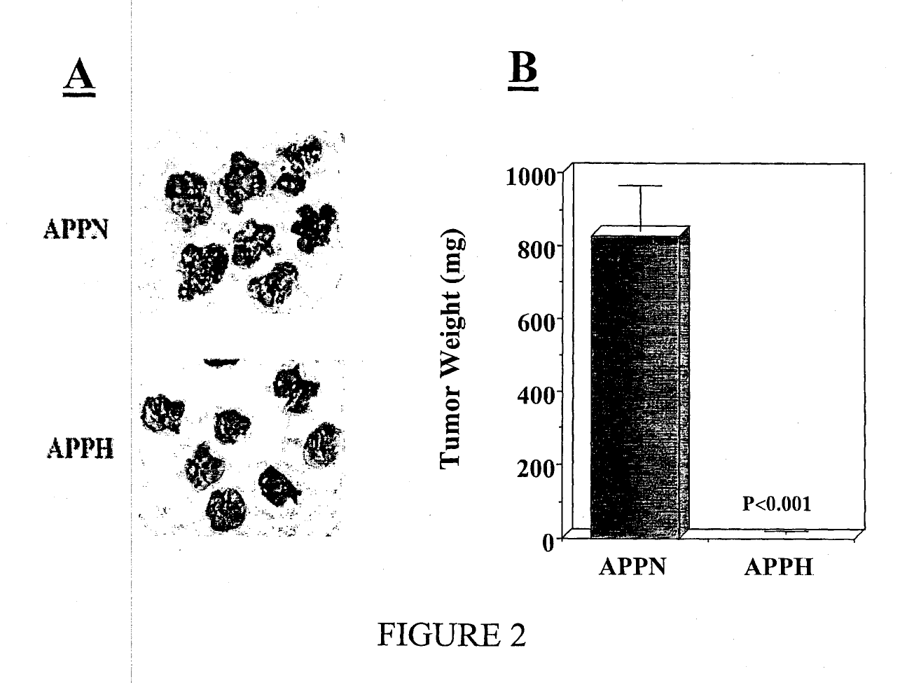

In another experiment lungs from Tg-APPH and control parental APPN mice were examined 37 days following i.v. injection of D122 tumor cells. Lungs from Tg-APPH mice were completely devoid of metastases, while in the control parental mice, two mice have died after 35 and 37 days and the rest of the mice were heavily loaded with metastases (820 mg) (FIG. 2A, 2B)

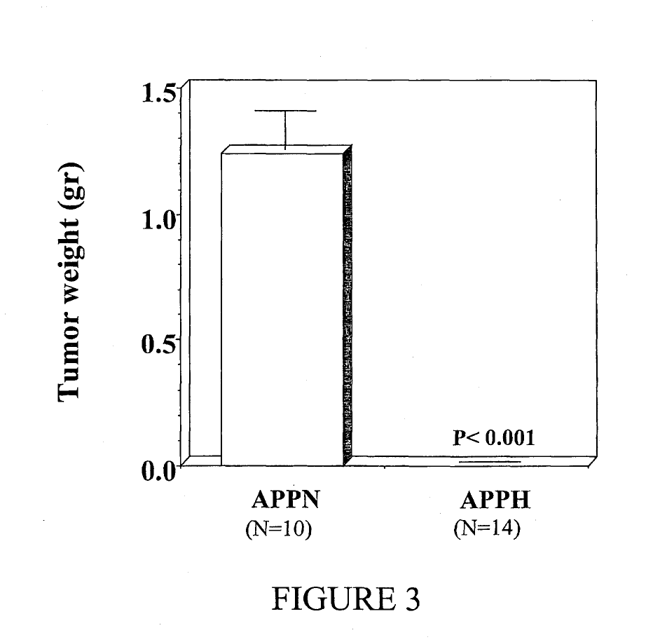

To examine the long-term effect of APP on metastasis, D122 tumor cells were injected i.v., and lungs were examined 136 days following tumor cell injection. No metastases whatsoever have developed in all 14 Tg-APPH mice even after 136 days following tumor cell injection. Similarly no metastases were found in any other organs of these mice. 13 out of 19 of the control parental APPN mice died after 17-36 days following tumor cell injection. All of the lungs of control APPN mice were heavily loaded with metastases (1240 mg) (FIG. 3).

### Example 2

B16 melanoma development is completely inhibited in Tg-mice. To examine whether the refractoriness of the transgenic mice to tumor metastasis is not specific to one type of tumor we have used the B16 melanoma model. B16 melanoma tumor cells were injected i.v. to the tail of female transgenic mice harboring the human APP gene (Tg-APPH) and to control parental mice (APPN). APPN control mice were examined after 23-27 days. All 16 mice were affected. Tumors developed in various organs in the APPN mice, such as: lungs, ovary, lymph nodes, kidneys and liver as described in Table 1. Many of the mice developed tumors in more than one organ. Tg-APPH mice were examined after 40-42 days. Fourteen out of the 16 mice did not develop any kind of tumor. Two out of 16 mice were affected. One tumor developed in the lungs and the other in a lymph node. The inhibition of tumor development in mice over-expressing APP, is not specific to one tumor type as well as it is not restricted to a certain organ target.

TABLE 1
Distribution of B16 Melanoma Tumors in APPN/APPH mice

| | APPN 23-27 days | APPH 40-42 days |
|---|---|---|
| Tumors | post injection | post injection |
| Tumor Incidence | 16/16 | 2/16 |
| Lung metastases | 9/16 | 1/16 |
| Ovary | 4/16 | 0/16 |
| Lymph | 7/16 | 1/16 |
| Kidney | 3/16 | 0/16 |
| Liver | 1/16 | 0/16 |

### Example 3

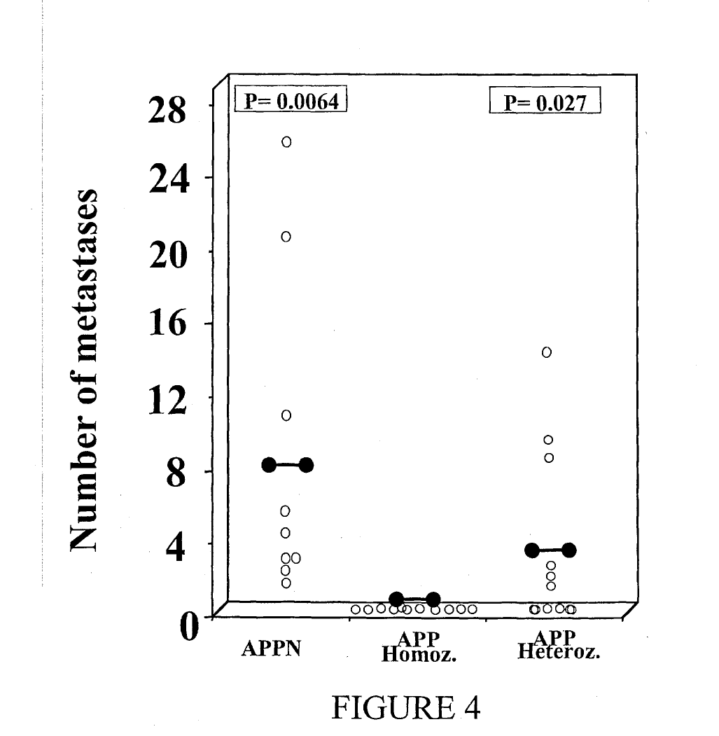

Heterozygous Tg-APP mice display an intermediate resistance to metastasis. To examine whether the effect of APP is dose dependent heterozygous APP mice, in which only one APP allele is expressed, were tested. Heterozygous APP mice were prepared by cross breeding Tg-APPH mice with control parental mice. D122 (84) tumor cells were injected i.v. into the tails of Tg-APPH, to APP-heterozygous mice and to control parental mice (APPN). After 35 days the lungs were examined and metastases were counted. No metastases whatsoever were found in the lungs of Tg-APPH. Metastases were found in the lungs of all control APPN mice. The average metastases number in control APPN lungs was eight (FIG. 4). On the other hand, only 6 out of 10 heterozygous Tg-APP mice had metastases in their lungs and the average metastases number was 3.5 (FIG. 4). These results demonstrate a correlation between level of APP gene expression and refractoriness to metastasis. APP has thus, a dose dependent effect on metastasis.

### Example 4

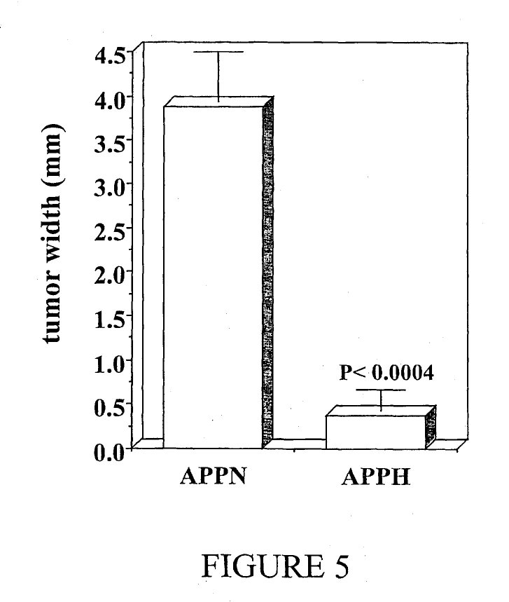

Lewis lung carcinoma and B16 melanoma primary tumors development is strongly inhibited in Tg-APP mice. Following the observation that the metastatic development is completely inhibited in Tg-APPH mice, we have examined whether primary tumor growth is also effected in Tg-APPH mice. Lewis lung carcinoma cells were injected into the footpad of Tg-APPH mice (APPH) and to control parental mice (APPN) for primary tumor development. The size of primary tumors in APPH and APPN mice was evaluated 24 days after injecting D122 tumor cells into the footpad by measuring the width of the footpad. Primary tumor development is inhibited in APPH mice by 90% as compared with control parental APPN mice, as shown in FIG. 5. Seven out of nine APPH mice did not develop any sign whatsoever of primary tumor even after 145 days following tumor cell injection. Similarly, no metastases were found in the lungs or in other organs of these mice 26 days following primary tumor removal.

Using the B16 melanoma model, similar results were obtained. Nine out of ten Tg-APPH mice did not develop any sign whatsoever of primary tumor even after 60 days following injection to the footpad. Similarly, no metastases were found in the lungs or in other organs of these mice 30 days following primary tumors removal.

### Example 5

Development of implanted tumors in Tg-APPH mice is slowed. Primary tumors did not develop in Tg-APPH mice when tumor cells were injected to the footpad. To examine the effect of APP on established tumors we implanted subcutaneously, Lewis lung carcinoma tumors previously grown in C57/BL mice into Tg-APPH and non transgenic control parental mice. The tumors grown in C57/BL mice were cut to 4 mm in diameter pieces and transplanted subcutaneously to transgenic and non-transgenic control parental mice. Tumors were removed from the backs of the mice after 27 days and weighed. Tumor weight in control parental mice was 2.73 gr.±0.43 S.E. (n=6) while in Tg-APPH mice was 1.34 gr.±0.26 S.E. (n=9) (p<0.013). A similar 3.3±0.54 S.E. fold decrease in tumor volume in the Tg-APPH mice as compared to that in control parental mice was measured. Moreover, histological sections prepared from these tumors demonstrate a 48%±3.64 S.E necrosis in the tumors derived from Tg-APPH mice as compared to only 5%±0.46 S.E necrosis in the tumors derived from control parental mice. This indicates that the actual mass difference between primary tumors derived from Tg-APPH mice is 4 fold smaller than that from control parental mice. In additions, at the time of primary tumors removal, metastases were found in the lungs of two out of six control parental mice, while no metastases were found in all 9 of the Tg-APPH mice.

### Example 6

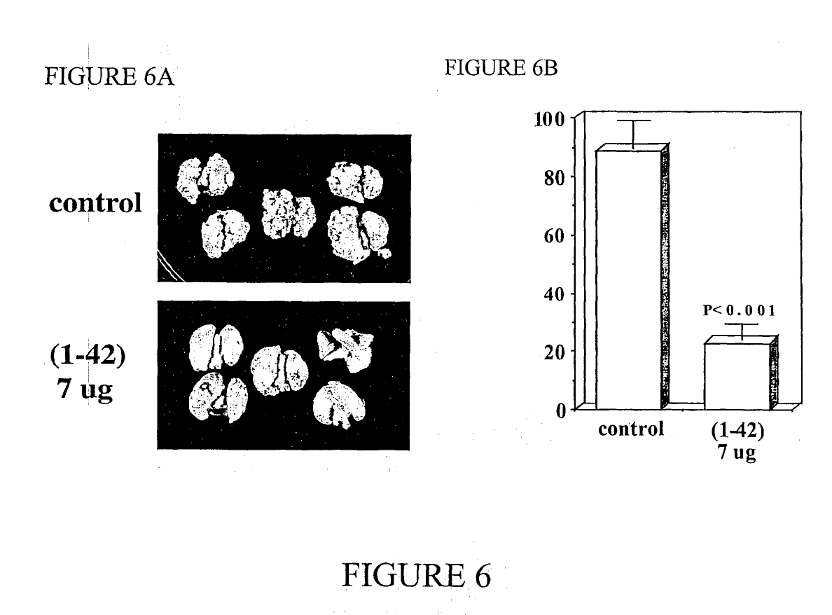

Effect of externally injected Aβ₁₋₄₂ peptide on metastasis in the lungs. To examine the effect of an externally added peptide which comprises a domain in the APP protein, we chose the Aβ₁₋₄₂ peptide. D122 tumor cells were injected i.v. to the tail of C57/BL mice. The Aβ₁₋₄₂ peptide (7 μg/mouse) was injected 4 times during the 10 days preceding D122 tumor cell injection and seven times after tumor cell injection. All together 77 μg of AP 1-42 were injected into each mouse. After 21 days tumor weight and number of metastases in the lungs were examined. Treatment with the AP 1-42 peptide reduced the number of metastases by 74% as demonstrated in FIG. 6A and FIG. 6B. Visual examination of various tissues (kidney, liver, lungs, brain and spleen) of Aβ₁₋₄₂ treated mice as well as histological sections prepared from these tissues revealed no pathological findings in the tissues.

### Example 7

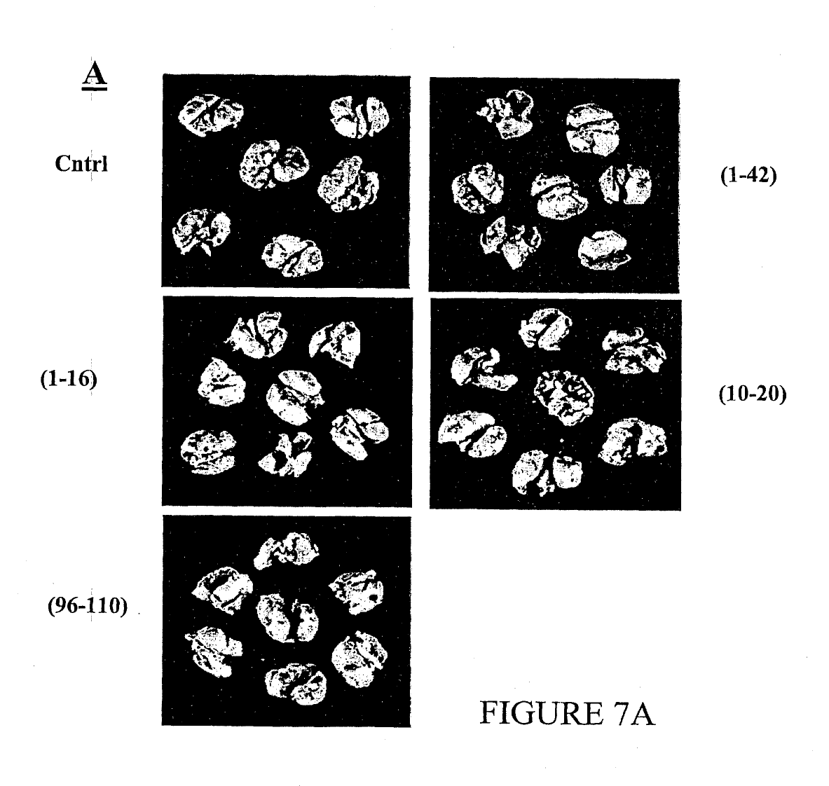

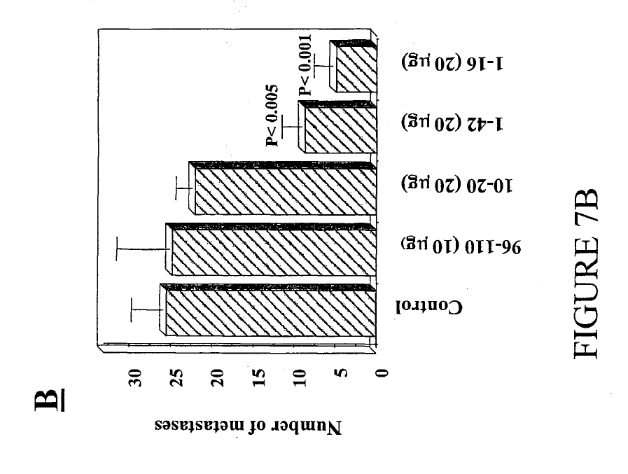

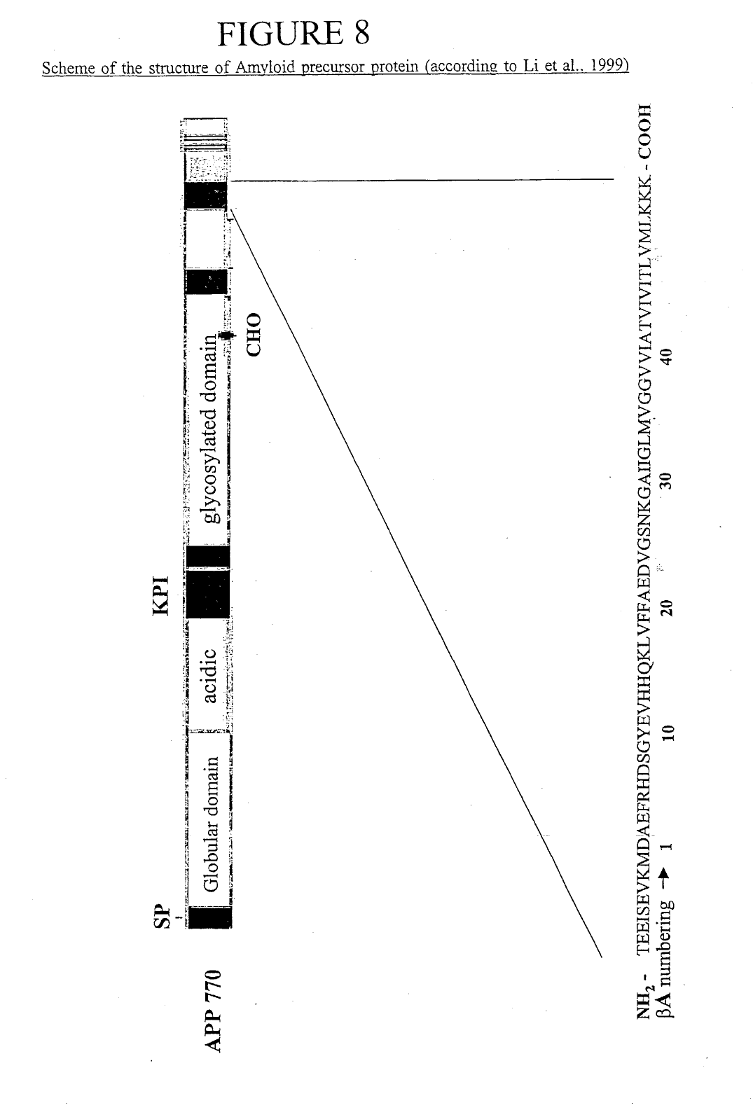

Effect of externally injected Aβ peptides on metastasis in the lungs. To further examine the effect of externally added peptides which comprise domains in the APP protein, we have used the Aβ₁₋₄₂, Aβ₁₋₁₆, Aβ₁₀₋₂₀ and Aβ₉₆₋₁₁₀ peptides. D122 tumor cells were injected intravenously to the tail of C57/BL mice. The AP 1-16, AP 1-42 and AP 10-20 peptides (20 μg/mouse/injection, respectively) or the Aβ₉₆₋₁₁₀ peptides (10 μg/mouse/injection) were injected 7 times during the two weeks preceding D122 tumor cell injection and seven times after tumor cell injection. All together 280 μg of Aβ₁₋₄₂, Aβ₁₋₁₆ and Aβ₁₀₋₂₀ and 140 μg of Aβ₉₆₋₁₁₀ were injected into each mouse. After 21 days the number of metastases in the lungs was examined. Treatment with the Aβ₁₋₁₆, Aβ₁₋₄₂, Aβ₁₀₋₂₀ and Aβ₉₆₋₁₀ peptides reduced the number of metastases in the lungs by 81%, 66%, 17% and 9% respectively, as demonstrated in FIG. 7A and FIG. 7B.

Most interestingly, the smaller peptide Aβ 1-16 which is non-toxic to neuronal cells was shown to be the most effective in inhibiting lung metastasis.

### Example 8

Exogenously added spleen cells from Tg-APP mice, decrease metastasis in wild type C57/BL mice. To examine whether inhibition of metastasis can be transferred, we have injected spleen cells from Tg-APPH mice into wild-type C57/BL mice. Injection of 20 million spleen cells (i.v.) to C57/BL mice 8 days before D122 tumor cells, decreased metastases number in the lungs of C57/BL mice by nearly 65%, as compared to non-treated mice. The number of metastases in non-treated mice was 15.3±16.6(S.D.), while number of metastases was reduced to 4.87±4.6(S.D.) in treated mice.

The mechanism or mechanisms by which the adoptive transfer of spleen cells from TgAPP mice prevents tumor spread or metastases in the host wild type mice is not yet fully elucidated. It may involve homing of the transferred cells to specific target organs, and/or it may involve induction of expression of APP or other effector molecules in the host. Without wishing to be limited to any particular mechanism of action, the fact that adoptive transfer of these cells confers enhanced resistance of the naïve hosts to tumor growth or metastasis is highly advantageous, since this is not by means of a permanent change in the genome.

This system serves as a clear indication that gene therapy may be performed with cells bioengineered to express APP, or APP-derived peptides. More advantageously the cells may be autologous cells of the individual who is in need of the treatment. By way of non-limitative example the cells may be Peripheral blood lymphocytes (PBL) or stem cells including but not limited to bone marrow derived stem cells of the patient. Alternatively and advantageously, gene therapy may be performed either in vivo or ex-vivo using other vectors or other cells types, by all of the methods well known in the art. It may also be possible to use human-embryonic stem cells for this purpose.

Moreover, since spleen cells constitute cells of the immune system, it is possible that the adoptive transfer of cells over-expressing APP activated the immune system, therefore, APP and APP-derived peptides or gene therapy with cells bioengineered to express APP or APP-derived peptides may be useful for treatment of immune deficiencies, such as occur in immune compromised individuals, for instance during or after chemotherapy.

### Example 9

Methods of Treatment with APP or APP-Derived Peptides

Hereinafter, the term “subject” refers to the human or lower animal to whom APP or APP-derived peptides are administered. The term “patient” refers to a human subject. The term treatment includes both substantially preventing tumor growth from starting, for instance in the case of metastatic tumors, and slowing or halting the progression of tumor growth once it has arisen. It is also possible to treat prophylactically high-risk individuals who are genetically or otherwise predisposed to development of cancer. The term “methods of treatment” include both treatment with the proteins or peptides of the invention as pharmaceutical compositions or gene therapy techniques.

Suitable Formulations for Administration of APP or APP-Derived Peptides

APP or APP-derived peptides can be administered to a subject in a number of ways, which are well known in the art. Hereinafter, the term “subject” refers to the human or lower animal to whom APP or APP-derived peptides is administered. For example, administration may be done parenterally, for example by intravenous drip or into the tumor site, as well as by intra-arterial, intrathecal, subcutaneous, or intramuscular injection.

Due to the peptide nature of the therapeutic agents disclosed herein it is not anticipated that oral bioavailability will be readily achieved. Nevertheless, it is within the scope of the present invention that certain formulations may provide or facilitate oral bioavailability of peptide therapeutics.

Formulations for parenteral administration may include but are not limited to sterile aqueous solutions which may also contain buffers, diluents and other suitable additives.

Dosing is dependent on the severity of the symptoms and on the responsiveness of the subject to APP or APP-derived peptides. The skilled attending physician can determine optimum dosages, dosing methodologies and repetition rates, as is known in the art.

Furthermore, it is well known in the art that it is possible to improve pharmacokinetics, stability or other desired properties of the peptides of choice by modifications of the termini, for example by addition of blocking groups, reduction or acylation and peptidomimetic linkages not found in naturally occurring peptides. All of these are explicitly encompassed within the scope of the present invention.

Method of Treatment of Cancer or Prevention of Metastasis

As noted above, APP or APP-derived peptides have been shown to be an effective inhibitor of tumor growth or metastasis. The following example is an illustration only of a method of treating cancer with APP or APP-derived peptides, and is not intended to be limiting.

The method includes the step of administering APP or APP-derived peptides, in a pharmaceutically acceptable carrier as described above, to a subject to be treated. APP or APP-derived peptides are administered according to an effective dosing methodology, preferably until a predefined endpoint is reached, such as the absence of further progression of tumor growth.

Examples of types of tumors for which such a treatment would be effective include, but are not limited to: bladder, brain, breast, cervix, colon, ear, esophagus, gastrointestinal, head and neck, kidney, larynx, liver, lung, myeloma, ocular, melanoma, ovary, pancreas, prostate, skin, stomach, thyroid, urethra, and uterus.

Additional malignancies for which such treatment would be effective include but are not limited to Kaposi's sarcoma, lymphoma, and leukemia.

Since metastasis is a serious complication of primary tumor growth all of these methods can also be used to treat or prevent metastatic tumors, in addition to treating or preventing those conditions characterized by primary tumors alone.

Method of Manufacture of a Medicament Containing APP or APP-Derived Peptides

The following is an example of a method of manufacturing APP or APP-derived peptides. First, APP or APP-derived peptides are isolated or synthesized in accordance with good pharmaceutical manufacturing practice. Examples of methods of isolating APP or synthesizing APP-derived peptides, are given in U.S. Pat. No. 5,550,216. Next, APP or APP-derived peptides is placed in a suitable pharmaceutical carrier, as described above, again in accordance with good pharmaceutical manufacturing practice.

It will be appreciated by the skilled artisan that due to the protein or peptide nature of the therapeutic agents disclosed herein it will also be appropriate to utilize recombinant DNA technology to obtain these agents in sufficient quantities to serve as raw materials for the pharmaceutical compositions.

Gene Therapy Using APP or APP-Derived Peptides

It will further be appreciated that it may be possible to utilize gene therapy strategies as a basis for effective therapy of the subject in need of treatment. Suitable potential vectors for the introduction of the gene encoding the APP or an active peptide derived therefrom are well known in the art. The use of gene therapy strategies for the introduction of anticancer molecules is exemplified for instance in Curiel D. T. (1999) and similar reference texts. Utilization of cells bioengineered to express the desired protein or peptide may be advantageous since it permits selection of the cells producing the desired molecules prior to their transfer to the subject in need of therapy. These cells can also be engineered to have inducible genes that will result in pre-programmed cell death if necessary.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

What is claimed is:

1. A pharmaceutical composition comprising a non-glycosylated peptide of an amyloid precursor protein and a pharmaceutically acceptable diluent or carrier.

2. The pharmaceutical composition of claim 1, wherein the peptide is derived from an AP domain of the amyloid precursor protein.

3. The pharmaceutical composition of claim 2, wherein the peptide comprises about 5 to 100 amino acid residues.

4. The pharmaceutical composition of claim 3, wherein the peptide comprises about 10 to 50 amino acid residues.

5. The pharmaceutical composition of claim 4, wherein the peptide comprises about 15 to 45 amino acid residues.

6. The pharmaceutical composition of claim 5, wherein the peptide is selected from the peptides having the sequence of peptides 1-43, 1-42, 1-16 and 10-20 of the Aβ domain of the amyloid precursor protein.

7. The bituminous composition of claim 3, wherein the second acid component is a strong acid component present in an amount of about 0.01 weight percent to 0.3 weight percent of the bituminous composition and the second base component is a weak base component present in an amount of about 0.1 weight percent to 2 weight percent of the bituminous composition.

8. A method for treating or preventing cancer or immunostimulation in a subject, which comprises administering to said subject a therapeutically effective amount of amyloid precursor protein.

9. A method for treating or preventing cancer or treating immunodeficiency in a subject, the method comprising administering to said subject a therapeutically effective amount of the non-glycosylated peptide of an amyloid precursor protein.

10. A method for treating or preventing cancer or treating immunodeficiency in a subject, the method comprising administering to said subject a vector comprising a polynucleotide construct encoding an amyloid precursor protein or a peptide derived therefrom.

11. The method of claim 10, wherein the treatment is ex vivo.

12. The method of claim 10, wherein the treatment is in vivo.

13. A method for treating or preventing cancer or treating immunodeficiency in a subject, the method comprising administering to said subject a pharmaceutical composition comprising cells comprising a polynucleotide construct encoding an amyloid precursor protein or a peptide derived therefrom.

14. The method of claim 13, wherein the cells are autologous cells.

15. The method of claim 14, wherein the cells are peripheral blood leukocytes.

16. A method of manufacture of a medicament for immunostimulation in a subject, comprising a step of using an amyloid precursor protein.

17. A method of manufacture of a medicament for immunostimulation in a subject, comprising a step of using a non-gylcosylated peptide.

18. A method of manufacture of a medicament for treating or preventing tumor growth or metastasis, comprising a step of using an amyloid precursor protein.


SEQUENCE LISTING (4 sequences)

SEQ ID NO: 1 - protein, 42 aa, Homo sapiens [sequence omitted]

SEQ ID NO: 2 - protein, 16 aa, Homo sapiens
DAEFRHDSGYEVHHQK

SEQ ID NO: 3 - protein, 11 aa, Homo sapiens
YEVHHQKLVFF

SEQ ID NO: 4 - protein, 15 aa, Homo sapiens
NWCKRGRKQCKTHPH